(19) United States
(12) Patent Application Publication
(10) Pub. No.: US 2005/0048039 A1
(43) Pub. Date: Mar. 3, 2005

(54) Method for the treatment or diagnosis of human pathologies with disseminated or difficult to access cells or tissues

(75) Inventors: Patrick A. Dreyfus, Clamart (FR); Elaine Parrish, Saint-Denis (FR); Luis Garcia, Saint-Denis (FR); Mohamed Chokri, Strasbourg (FR); Jacques Bartholeyns, Bures-Sur-Yvette (FR); Elise Peltekian, Paris (FR)

Correspondence Address: YOUNG & THOMPSON, 745 SOUTH 23RD STREET, 2ND FLOOR, ARLINGTON, VA, 22202, US

(73) Assignees: INSTITUT NATIONAL DE LA SANTE ET DE LA RECHERCHE MEDICALE (I.N.S.E.R.M), PARIS CEDEX 13 (FR); I.D.M. IMMUNO-DESIGNED MOLECULES, PARIS (FR)

(21) Appl. No.: 10/766,929

(22) Filed: Jan. 30, 2004

Related U.S. Application Data
(62) Division of application No. 08/924,830, filed on Sep. 5, 1997, now abandoned.

(51) Int. Cl. A61K 48/00

(52) U.S. Cl. 424/93.21

(57) ABSTRACT

Method for the treatment or diagnosis of pathologies either expressed in injured or pathological multiple sites in tissues or in the body or expressed in injured or pathological sites of tissues or cells in sites of the body, which are difficult to access, with said sites or areas in immediate proximity to said sites being the source of the release of chemotactic factors for endogenous macrophages, either spontaneously or upon suitable stimulation, wherein said treatment is carried out by administration to the body of an appropriate amount of exogenous monocyte derived cells, said monocyte derived cells being, in the case of treatment, loaded with corrective agents with respect to the pathologies to be treated, and with said monocyte derived cells having the properties of mobilisation towards the source of the above-said released chemotactic factors and in target the cells present in the vicinity of the said released chemotactic factors, and in the case of diagnosis, loaded with a marker enabling the detection of injured or pathological sites.

The invention relates to an original procedure to simultaneously target disseminated or difficult to access pathological sites, and to deliver a therapeutic agent or an agent exerting a therapeutic activity everywhere it is required for the purpose of treating human diseases more generally mammals.

The invention also relates to ex-vivo prepared monocyte derived cells as in vivo therapeutic vectors enabling the precise and specific targeting of affected cells or tissues.

The invention also relates to pharmaceutical compositions containing said monocyte derived cells.

Gene therapy as a treatment for, amongst others, inherited diseases and cancer, is an ever developing concept based on the use of DNA as the therapeutic agent. For any given disease, obtaining an adequate therapeutic gene is a prerequisite, although only the beginning of a multi-step process, encompassing the appropriate vectorisation of this gene and the successful targeting of all affected sites. Gene therapy for solid tissues has, so far, dealt with injections of recombinant viral vectors (Quantin et al., 1992; Ragot et al., 1993; Vincent et al., 1993), preparations of naked DNA (Wolff et al., 1990; Acsadi et al., 1991), or lethally processed murine packaging cells (Fassati et al., 1995) directly into the affected tissues. This delivery technique is, nevertheless, of limited clinical use in diseases characterised by a widespread distribution of, and/or difficult access to, the pathological sites.

The possibility of using transplanted immortalised monocyte-like murine cells has previously been demonstrated, with cells transformed using the SV40 T antigene, as naturally homing shuttles able to target multiple disseminated lesions in skeletal muscle diseases. These cells, injected directly, intravenously, into mice successfully attained experimentally induced necrotic muscle sites showing that a one-off administration of cells can rapidly target a given pre-existing muscle injury and probably any inflammatory zone (Parrish et al., 1996).

One of the aims of the invention is to provide with circulating monocyte derived cargo-cells (also termed macrophages, phagocytes, mature phagocytes, monocyte derived cells loaded), capable of homing subsequently to the widespread distribution sites and/or difficult access sites and to deliver appropriate therapeutic agent.

Another aim of the invention is to provide with relevant tools to deliver therapeutic genes or drugs into injured tissues, particularly the central nervous system, or sites releasing factors chemotactic for macrophages or for monocyte derived cells.

According to an advantageous embodiment, the invention relates to a method for the treatment or diagnosis of pathologies.
either expressed in injured or pathological multiple sites in tissues or in the body,
or expressed in injured or pathological sites tissues or cells in sites of the body, which are difficult to access,

with said sites or areas in immediate proximity to said sites being the source of the release of chemotactic factors for endogenous macrophages, either spontaneously or upon suitable stimulation, wherein said treatment is carried out by administration to the body of an appropriate amount of exogenous monocyte derived cells,

said monocyte derived cells being:
in the case of treatment, loaded with corrective agents with respect to the pathologies to be treated, and with said monocyte derived cells having the properties of mobilisation towards the source of the above-said released chemotactic factors and to target the cells present in the vicinity of the said released chemotactic factors,
and in the case of diagnosis, loaded with a marker enabling the detection of injured or pathological sites.

The expression “exogenous monocytes derived cells” corresponds to cells differentiated ex vivo by culture of blood monocytes and charged with chemical or biological substances or transfected with a virus to vectorize these elements towards injured areas of the body. In the following these monocyte derived cells will also be called “monocyte derived cargo cells”.

By “multiple sites” is meant, for instance,
metastatic tumor cells troughout the body or a tissue
general inflammation of joints such as arthritis
widespread sites of tissue injury or degeneration, such as numerous lesions in multiple sclerosis.

The expression “sites difficult to access”, corresponds to sites which cannot be reached easily by local or systematic injection, such as the CNS (central nervous system) which is segregated by the blood brain barrier or such as necrotic areas, bones or eyes.

The expression “chemotactic factors” corresponds to chemokines or factors released in injured sites or areas (in particular by suffering or dead cells) which attract specifically macrophages which present receptors sensitive to said chemotactic factors and move to area where the concentration of chemotactic factors is higher than in the immediate vicinity of said macrophages. Endogenous macrophages are responding locally in so far as they are present in the injured areas, but are not present in the blood stream, in contrast to the monocyte derived cells of the invention.

The injured sites or areas in immediate proximity to said sites, which are the source of the release of chemical factors will be called in the following “sites of call”. It is to be noted that the sites of call always contain pathological or injured sites and also non injured and non pathological by stander cells.

The immediate proximity to an injured site is defined as the cells which are within less than 10 mm from the injured site.

The monocyte derived cells used in the method of treatment of the invention can be or not loaded with corrective agents, and are preferably loaded with corrective agents.

The expression “corrective agent” means correspond to a chemical or biological substance or virus carrying a gene for such substance which can have a benefit on the treatment or the pathology.

For instance, in case of a genetic deficit, the corrective susbstance corrects the deficit by enzyme replacement; in case of cancer, the corrective substance kills tumor cells;

in case of neuromuscular degenerescence the corrective substance is a factor for protection or regeneration.

The expression “mobilisation” corresponds to a chemotactism (diapedesis) towards the sites of cells were the signal originates and to the accumulation of the monocyte derived cells of the invention around this site.

The term “target” means that the monocyte derived cells of the invention affects specifically the cells present in the vicinity of the site of call.

As to the body, it is meant the animal or preferably the human body.

Preferred applications are on the human being.

In the case of diagnosis, the marker is preferably a dye or a radiation emitting substance. This diagnostic methods can be used to detect sites of early metastatic development or undected sites of cranial trauma or injuries. This diagnostic method can be proposed prior to a treatment according to the invention or prior to an unrelated treatment (surgery, etc. . . ).

In an advantageous embodiment of the invention, the treatment with said corrective agents consists in providing deficient elements, such as those responsible for or resulting from the pathology, or providing elements liable to inhibit or to kill abnormally stimulated cells, responsible for or resulting from the pathology.

By way of example, a “deficient” element can be an enzyme or protein or growth factor which is missing in genetic diseases or after degeneration/senescence.

Elements liable to “inhibit abnormally stimulated cells” can for instance:
inhibit proliferation of tumor cells;
inhibit the release of cytokines and inflammatory factors;
relieve the chronic stimulation of muscles or nerves;
inhibit angiogenesis,

In an advantageous embodiment of the invention, the corrective agent is a chemical or a biological product such as a polypeptide, a growth factor, a nucleic acid, a gene or the product of a gene.

In an advantageous embodiment of the invention, the monocyte derived cells are prepared ex vivo by culturing blood monocytes to obtain monocyte derived cargo cells and in particular mature phagocytes and loading said cells with appropriate chemical or biological substances and enhancing their capability (signal linked to the membrane, carrier of product or information, phagocytosis and secretion) or/and transfecting them with a virus containing an appropriate gene of or with nucleic acids consisting in or containing an appropriate gene.

By “mature phagocytes” are meant phagocytes (for example macrophages) differentiated from monocytes, which do no proliferate and actively digest external element (marker CD68, HLADR, mannose receptor).

The appropriate gene corresponds to a gene which, if deficient, will cause the disease.

In an advantageous embodiment of the invention, the chemotactic factors are released either by injured or pathological sites spontaneously resulting from the pathology or subsequent to a chemical or physical stimulation of the sites to be treated.

The expression “chemical or physical stimulation” for instance means radiation, chemotherapy, peptide or toxin injection, puncture, local freezing . . . causing local injury and release of the chemotactic factors (signal).

The inducted stimulation will create directly or indirectly the signal for monocyte derived cells to proceed and fix to the site of call.

The chemical signal can preferably be given by injection of drugs, toxins, antibodies recognizing the target cells, hormones, excitory amino acids, detoxified endotoxins or antigens.

The physical signal can preferably be given by local irradiation, cryoburning, laser, local release of cytotoxic or chemotactic factor, microsurgery.

In an advantageous embodiment of the invention, the multiple expressed sites result from disseminated cancers or from inflammatory diseases.

The expression “disseminated cancer of inflammation” means cancer or inflammation present in multiple sites/organs of the body or present only in one organ or tissue, but an multiple spots rather than at a defined area.

In an advantageous embodiment of the invention, the injured or pathological sites difficult to access are: the central nervous system, the peripheral nervous and muscular systems and bones.

The “central nervous system” designates classically brain, cerebellum, spinal cord segregated from blood and the penetration of most substances by the blood brain barrier.

The “peripheral muscular nervous system” classically designates the nervous system localized in peripheric tissues, where there is access but in which it is difficult to target only the injured area.

By way of example the pathologies to be treated include:
For the central nervous system
   Genetic diseases such as:
      Adrenoleukodystrophy
      Spinal muscular atrophy
      Gaucher disease
      Huntington disease
   Sporadic diseases such as:
      Alzheimer disease
      Parkinson disease
      Amyotrophic lateral sclerosis
      Multiple sclerosis
      Strokes
      Glioblastoma
      Cerebral metastasis
      Infection of the central nervous system
Peripheral nervous and muscular system
   Genetic diseases such as:
      Duchenne disease, Becker disease
      Muscular dystrophies
   Non genetic diseases such as:
      Neuropathies and muscular necrosis from different origins (incl. trauma)
Rheumatoid arthritis
Atheromatosis
Bone trauma or bone infection or degenerescence
Pulmonary fibrosis.

The invention also relates to monocyte derived cells obtained by culturing blood mononuclear cells to obtain monocytes derived cargo-cells, containing a therapeutic agent for a given pathology corresponding to loaded chemical or biological substances such as peptides, polypeptides, proteins and nucleic acids or to virus or nucleic acids which have been transfected into said cells or to these cells loaded externally on the membrane with emitting signals, the said cells having one or more of the following properties:
their preparation specifically induce an increased membrane expression level of chemotactic receptors,
they are sensitive, particularly in vivo, to chemotactic factors released by sites of call or suffering cells,
they have membrane a plasticity such that they can enter difficult injured sites to access such as the central nervous systems,
they can rapidly reach sites of call, as soon as two hours to three days, particularly two to three days after systemic injection,
they can accumulate into injured sites of call,
they remain alive in the vicinity of the injured or pathological sites for several months, particularly at least up to about 4 months,
their morphology becomes similar to the morphology of the cells normally present in the injured sites or pathological and they integrate the tissue cells of the injured or pathological sites,
they can release the contained corrective agent in the sites of call, either constitutively or on demand by induction of secretion of said corrective agent.

The monocyte derived cells of the invention present also the following properties: they cannot divide and they can phagocyte macromolecular particles or debris.

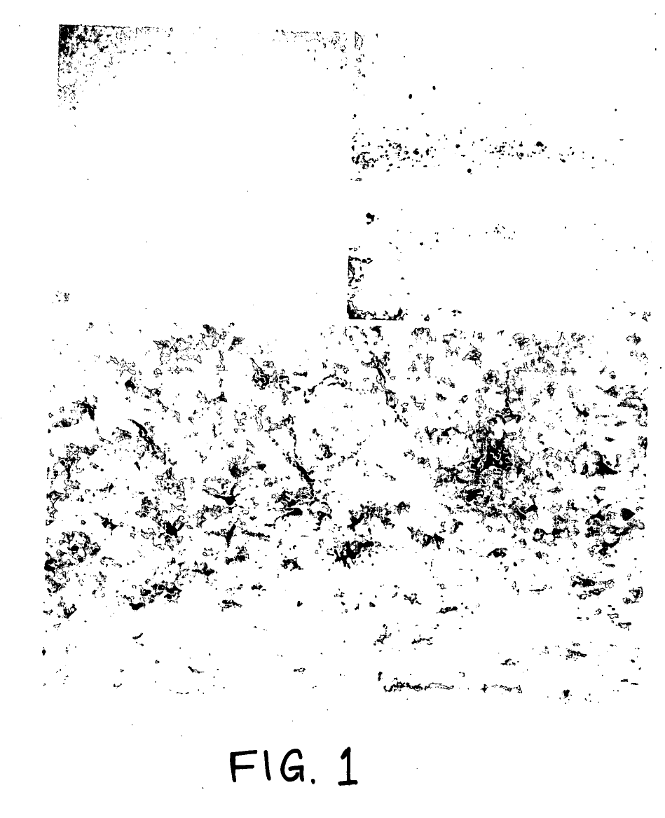

All these properties can be checked according to the experiment described in the example section and concerning the feasibility of targeting a central nervous system lesion with exogenous engineered monocyte derived cells (see FIG. 1).

The concentration of chemotic factors to which the monocyte derived cells are sensitive can be as low as 10⁻¹² M.

The plasticity property corresponds to the fact that the monocyte derived cells of the invention can migrate into most extravascular spaces.

According to an advantageous enbodiment, the monocyte derived cells according to the invention are loaded with chemical or biological substances introduced either by phagocytosis, pinocytosis or physical means such as electropulsation.

The “phagocytosis” corresponds to an interiorisation or particles by engulfment and endocytosis requiring energy and reorganisation or cytoskeleton.

The “pinocytosis” corresponds to a fluid phase endocytosis relatively passively.

The “physical means” such as electropulsation corresponds to a reversible change in membrane potential allowing interiorisation or drugs/compounds present in the extracellular fluid and which normally do not or slowly cross the membrane.

In an advantageous embodiment of the invention, the monocyte derived cells are transduced using different defective viral vectors such as adenovirus, herpes simplex virus and lentivirus, thereby allowing the transduction of said monocyte derived cells to efficiently introduce therein a cassette containing nucleic sequences coding for a secretable therapeutic peptide, polypeptide or protein under the control of a specific promoter such as Pz.

In an advantageous embodiment of the invention, the monocyte derived cells are transfected by introduction of a viral construction consisting of both a murine leukemia provirus (MuLV) containing a gene encoding a peptide, a polypeptide or protein of therapeutic interest and sequences encoding the helper genome allowing its mobilisation and the release of the viral construction at the injured sites.

These packaging MDC cells will release viral particles at the site of injury or at the site where the signal (chemotactic factors) is delivered. Preferably retroviruses will be used for proliferating target cells, while lentiviruses, adenoviruses, herpes viruses or canaripoxviruses will be used to infect postmitoric non proliferating target cells.

In an advantageous embodiment of the invention, the monocyte derived cells are
either transduced sequentially with:
   a) a defective viral vector (matrix vector), able to transduce post-mitotic cells, carrying the sequences encoding entirely the provirus defined above (which carries the therapeutic gene),
   b) a defective viral vector (assembling vector), able to transduce post-mitotic cells, carrying a defective MuLV gag-pol-env genome for transcomplementation allowing replication of the above-said provirus,
or transduced by a single defective viral vector (master vector), able to transduce post-mitotic cells, carrying both the sequences encoding entirely the provirus defined above (which carries the therapeutic gene under the control of an internal promoter Py) and a defective MuLV gag-pol-env genome under the control of an internal promoter Pz, for ciscomplementation allowing replication and production of the above-said provirus.

The gene of interest carried by the matrix vector in the sequential transduction or by the master vector in the one step viral transduction will preferentially be a gene encoding a suicide molecule, a growth factor, an ion channel protein, a metabolic protein, a structural protein, a transcriptional protein, or an antisens sequence allowing suppression of gene expression or exon skipping.

The invention also relates to a kit for the preparation of monocyte derived cells according to the invention comprising one or more of the following components:
culture means (bags and means) for the maturation of mononuclear cells into phagocytes, particularly macrophages,
therapeutic agents to be introduced into the above-said phagocytes and means of introducing them to obtain monocyte derived cells.

The invention also relates to a kit as above defined containing one ore more of the following components:
means for viral transduction of said phagocytes with defective viral vectors to obtain monocyte derived cells,
description of physical (laser, puncture, irradiation . . . ) and chemical means to induce the local signal when required, including the time schedule,
reagents for the quality control of the viral transduction and of the monocyte derived cells.
software for the standard operating procedures and traceability particularly of the following steps: cultures of phagocytes, introduction of corrective agents, viral transduction and the recovery of the above-mentioned monocyte-derived cells.

The invention also relates to pharmaceutical compositions containing as active substance monocytes derived cells according to the invention in association with a pharmaceutically acceptable vehicle.

The appropriate amount of monocyte derived cells of the invention is administated preferably in an amount of about 10⁶ to about 10¹⁰ and preferably about 10⁷ to about 10⁹ monocyte derived cells for a therapeutic administration on an adult patient.

All these aspects have been achieved through means to produce tissue macrophages or monocyte derived cells from human monocytes. Said macrophages can be non activated macrophages such as those grown in defined medium from monocytes, without addition of exogenous cytokines. Said monocyte derived cells can be obtained in culture from monocytes after induction of membrane expression of chemotactic receptors. For instance activated macrophages can be obtained as described in Patents A61C 12N: 9001402; PCT EP 93 01232; PCT FR 96 00121; 96 401 0995. After 5 to 7 days in culture, primary monocytes lose some functions and makers (peroxidase activity, galactose receptors) and gain specific tissue macrophage properties and receptors (esterase activity, mannose receptor, CD64, CD68, tissue adhesins). These ex-vivo differentiated macrophages respond very effectively and rapidly to low concentrations of chemotactic factors, and due to their unique plasticity can migrate into most extravascular spaces very easily. They also present a very high phagocytic/pinocytic activity and can be charged with therapeutic agents, growth factors and nucleic acids, taken up actively or after transfection (viral, chemical or electroporation).

A single inflammatory episode, the presence of cell suffering or of an induced signal, therefore, triggers the implantation of a stable “reservoir” of therapeutic cells, and in so doing primes the area with constitutive or inducible emitters of beneficial factors, in a zone susceptible to further sporadic or progressive pathological evolution.

Thus, the MDC-cells exist in two forms:
i) “patrollers”, which can be summoned on demand, in acute reaction to already degenerating regions, or sites of call
ii) “sleepers”, which after stable colonisation of the targeted tissue, can act either on demand by induction of secretion of the therapeutic agent, or chronically by its constitutive production. These two approaches might well determine the use of multiple therapeutic factors, their secretion being governed by the particular state of differentiation of the MDC-cells of the invention.

The recruitment of MDC-cells into a defined site or tissue can also, when needed, be induced locally by physical means (radiotherapy, laser) or by local microinjection of chemotactic factors (detoxified LPS, chemokines), or systemic injection of a substance (in particular an antibody) which will accumulate in a site of call.

Engineering Procedure of MCD-Cells

MCD-cells will exist in two forms: “packaging MDC-cells” and “secreting MDC-cells”
Secreting MDC-cells consist of cells, prepared ex-vivo as previously described, either preactivated or charged with: i) drugs or growth factors, or ii) transduced using different defective viral vectors (adenovirus, herpes simplex virus, lentivirus) allowing the transduction of a post-mitotic cell to efficiently introduce a cassette containing sequences coding for a secretable therapeutic factor under the control of a specific promoter Pz.
i) Monocytes derived cells can be loaded internally with agents (drugs, growth factors, nucleic acids, chemicals or informations) or externally by linking to their membranes specific molecules being or emitting a signal such as adhesins, antibodies or radioligands. Loading can be achieved by phagocytosis (mediated or not by receptors), pinocytosis or by facilitation of the transport accross the cell membrane by physical means such as for example electropulsation or by direct interaction with cell membrane.
ii) Transduced cells to obtain secreting monocyte derived cells is described in Example 2
Packaging MDC-cells are created by introduction of both a murine leukemia provirus (MuLv) containing the gene encoding the therapeutic agent, and the sequences encoding the helper genome allowing its mobilisation. This is achieved in one of two ways which are described in Example 3

Range of Application

MDC-cells will not only naturally phagocytose debris, release monokines and growth factors in targeted areas, but in addition, will release the drug or the gene product for which they have been engineered. They can be used for the treatment of chronic or acute injuries, including genetic disorders of tissues difficult to access, such as the CNS. Autologous MDC and particularly macrophages will be preferentially used, but for immunoprotected areas, such as the brain, effective targeting and long lasting homing can be obtained with allogenic or xenogenic macrophages, or even cell lines. This would be of interest in acute situations such as “stroke” when there is no time for preparation of autologous MDC-cells.

MCD-cells are applied to two categories of treatment by gene therapy:
i) Anti-tumoral strategies (ablative) using either “secreting MDC-cells” releasing for example cytokines or factors affecting the growth of the tumor and boosting other treatments such as immunotherapy, or “packaging MDC-cells” releasing retroviral vectors carrying a suicide gene around proliferative tumor cells.

e.g. Glioblastoma (systemic injection(s) of MDC-cells, reaching the brain tumor at its most invasive periphery): x can be the suicide gene TK under the control of glial cell promoter such as GFAP (Py), gag-pol-env genome can be under the control of either on inducible or constitutive promoter (Pz).

ii) Corrective strategies: phenotypic compensations using “secreting MDC-cells” releasing a soluble factor, or genetic correction using “packaging MCD-cells” to release a corrective retroviral vector.
Degenerative diseases such as: spinal muscular atrophy, amyotrophic lateral sclerosis, Alzheimer's disease, adrenoleukodystrophy, Gaucher disease, muscular dystrophies (Duchenne), Huntington disease, Parkinson disease.

e.g. Amyotrophic lateral sclerosis (systemic injection(s) of MDC-cells, reaching the spinal cord via natural turn-over): x can be the CNTF (ciliary neurotrophic factor) gene under the control of a differentiation dependant or inducible promoter such as CD68 or the erythromycin inducible, respectively (Py).

e.g. Duchenne muscular dystrophy (systemic injection(s) of MDC-cells, reaching widespread sites of skeletal muscle necrosis/regeneration): x can be the mini-dystrophin or the utrophin gene under the control of muscle promoter such as desmin or dystrophin itself (Py), gag-pol-env genome can be under the control of either a macrophage differentiation dependant (such as CD68 or CD36) or an inducible promoter such as the erythromycin inducible (Pz).
Inflammatory diseases: multiple sclerosis, rheumatoid arthritis.

In conclusion, MCD-cells can be applied to any pathology where the stimulation suffrance or death of individual or groups of cells induces the recruitment of macrophages.

FIG. 1: represents the feasibility of targeting a central nervous system lesion with exogenous engineered monocyte derived cells (“therapeutic shuttles” or “cargo cells”) injected intravenously.

FIG. 2: represents a construction for the transduction of monocyte derived cells, particularly macrophages, comprising a defective viral vector (represented by ad (=adenovirus), HSV (=herpes sample virus) or lenti (=lentivirus) and a cassette containing sequences coding for a secretable therapeutic factor (x) under the control of a specific promoter (Pz).

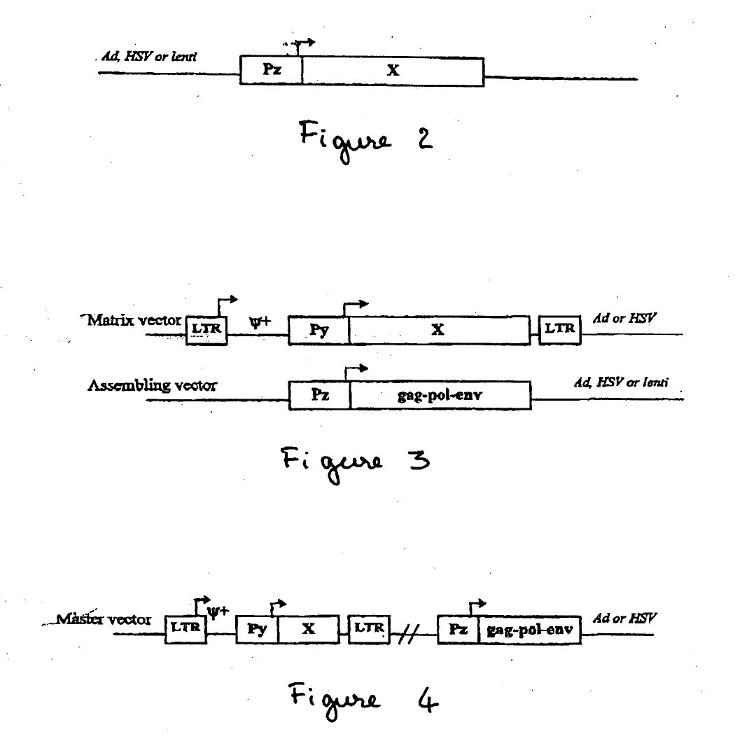

FIG. 3: represents constructions used for the sequential transduction of monocyte derived cells comprising
a matrix vector (represented by Ad (=adenovirus) or HSV (herpes simplex virus), with two long terminal repeats (LTR), a signal for packaging (Ψ⁺) and a gene of interest (X) under the control of an internal promoter (Pz),
an assembling vector represented by Ad, HSV or Lenti (corresponding respectively to adenovirus, herpes simplex virus or lentivirus) containing the sequences encoding gag, pol, and env genes from MuLV under the control of an internal promoter (Pz).

FIG. 4: represents a construction for the transduction of monocyte derived cells, comprising a single viral vector (master vector) carrying both:
a) the sequences encoding entirely the provirus (carrying the therapeutic gene X under the control of Py), with two long terminal repeats (LTR) and a signal for packaging (Ψ⁺), and
b) a defective MuLV gag pol env genome under the control of a promoter of Pz.

### EXAMPLE 1

Targeting a Central Nervous System Lesion with Engineered Monocyte Derived Cells

The feasibility of targeting a central nervous system lesion has been verified by injecting these cells intravenously into rats having previously received an intracerebral injection of kainic acid (methods are described in FIG. 1). This is a classic model of experimentally induced neuronal depletion in the rat, whose extent and chronology has been well documented. Schematically, neurones and astrocytes die rapidly within a few hours, oligodendrocytes disappear within a few days, and a cell halo enriched in macrophages-microglial cells appears after 2-3 days.

Sprague-Dawley male rats, weighing 250 g, (R. Janvier, France) were anaesthetised by intra-peritoneal injection of 3 l of a 4% solution of chloral hydrate (170 mg/kg) and positioned in a stereotaxic instrument (Stoelting). An incision was made along the midline of the scalp and hole drilled to allow injection on the right side of the brain using a Hamilton syringe. The stereotaxic coordinates for intra-striatal injections of 1 μl of a 10⁻³ M kainic acid solution were: anterior to the Bregma +1.2, lateral to the sagittal suture +2.3, ventral to the surface of the brain −4.5, according to Paxinos and Watson (1982).

Animals received a single caudal vein injection of 3×10⁶ MDC-cells 2 to 3 days after kainic acid lesion. Two days later, rats under deep anesthesia (sodium pentobarbital 45 mg/kg, i.p.) were perfused intra-cardially with 400 ml of phosphate buffered saline (PBS—pH 7.4), followed by 400 ml of 4% paraformaldehyde. Brains were subsequently dissected and placed in 30% buffered sucrose (pH 7.4) at 4° C. for 48h before freezing for histology. Perfused fixed brains were then frozen in dry ice cooled isopentane at 40° C. and 36 μm frontal sections were cut at =22° C. throughout the entire lesion. For specific detection of the human macrophages by immunocytochemical staining, floating sections were incubated for 1 hr in PBS, 0.3% Triton X-100, plus 5% normal horse serum, followed by incubation overnight at room temperature in PBS, 0.3% Triton X100 containing a mouse monoclonal antibody against HLA-DR (Dako) diluted 1/100. After rinsing three times in PBS, 0.3% Triton X100, sections were incubated for 1 hr in PBS, 0.3 Triton X100 containing a biotinylated horse anti-mouse antibody (Vector), diluted 1/200 in PBS, 0.3 Triton X100. Following rinsing in PBS, 0.3% Triton X100, incubation for 1 h at room temperature with a streptavidin-horseradish peroxidase complex (Vector, AB complex 1/300 in PBS, 0.3 Triton X100) and thorough rinsing in PBS, immunoreactivity was revealed using the Vector Peroxidase 3,3′-diaminobenzidine tetrahydrochloride (DAB)/DAB-nickel substrate kit. After staining, all sections were dehydrated in graded alcohol and toluene and mounted in Permount (Fisher Scientific).

Conclusion:

Examination of brain sections demonstrated a significant recruitment of the exogenous cells in and around the lesion zone (upper right of FIG. 1), with none observed in the healthy contralateral region (upper left of FIG. 1). Importantly, high magnification of the damaged area showed ramified cells clearly implanted in the parenchyma and not restricted to perivascular regions (lower of FIG. 1).

Human MCD and particularly macrophages accumulate into injured sites and not healthy sites of the brain. The macrophages injected home and acquire the characteristics of brain tissue cells, they remain alive at the CNS injured site for months.

### EXAMPLE 2

Preparation of Secreting Monocyte Derived Cells

Transduction of macrophages by viral vectors is achieved in suspension in a defined medium (RPMI) without serum. 4×10⁶ cells in 2 ml are incubated at 37° C. for 2 h with 4×10⁷ pfu of virus. Subsequently, after centrifugation (1000×g; 5 min), excess virus is removed followed by two successive washes in 500 ml of defined medium (RPMI) without serum. Cells are finally recovered in an appropriate volume and buffer for injection.

### EXAMPLE 3

Preparation of Packaging Monocyte Derived Cells

i) Macrophages are sequentially transduced with:
a) a defective viral vector (matrix vector), able to transduce post-mitotic cells, carrying the sequences encoding entirely the provirus (which carries the therapeutic gene);
b) a defective viral vector (assembling vector), able to transduce post-mitotic cells, carrying a defective MuLV (murine leukemia virus) gag-pol-env genome transcomplementation allowing replication and production of this provirus.

The matrix vector is a defective adenovirus, a defective herpes simplex virus or an amplicon. The provirus contains two LTRs (long terminal repeats), a signal for packaging (Ψ⁺), and a gene of interest (x) under the control of an internal promoter (Py). The assembling vector is a defective adenovirus, a defective herpes simplex virus, an amplicon or a defective lentivirus, containing the sequences encoding gag, pol, and env genes from MuLV under the control of an internal promoter (Pz).

ii) Macrophages are transduced by a single defective viral vector (master vector), able to transduce post-mitotic cells, carrying both the sequences encoding entirely the provirus (which carries the therapeutic gene under the control of Py) and a defective MuLV gag-pol-env genome under the control of Pz, for ciscomplementation allowing replication and production of this provirus.

Gene of Interest:

X can be a gene encoding a suicide molecule, a growth factor, an ion channel, a metabolic protein, a structural protein, a transcriptional protein, or an antisense sequence allowing suppression of gene expression or exon skipping.

Control:

Py can the provirals 5′LTR itself, a constitutive promoter such as another viral promoter (e.g. CMV, RSV, SV40) or a house-keeping gene, an inducible promoter, or tissue specific promoter. Pz can be the proviral 5′LTR itself, a constitutive promoter such as another viral promoter (e.g. CMV, RSV, SV40) or a house-keeping gene, an inducible promoter, or differentiation dependant promoter (e.g. CD68; CD36).

### EXAMPLE 4

A human bearing a brain degenerative disease is injected intravenously with monocyte derived cargo cells (10⁹) loaded with a growth factor according to the invention.

Human having a central nervous system degenerative disease is treated by intravenously injected monocyte derived cargo-cells (10⁹) secreting a neurotrophic factor.

The potent effect of ciliary neurotrophic factor (CNTF), GDNF (glial derived cell neurotrophic factor) and cardiotrophin 1 on motoneuronal survival is extensively documented. For example a patient suffering of ALS (amyotrophic lateral sclerosis) can be treated by CNTF locally delivered in the microenvironment of motoneuronal degeneration. In an animal model of ALS disease (Lou Gehring disease) it has been demonstrated a 3 fold increase of the microglial cells (brain macrophages) surrounding (forming an array) the suffering motoneurons. It has been shown that at least 50% of brain macrophages are recruited from blood borne cells.

The monocytes from the patient are collected by cytapheresis, and ex vivo differentiated into macrophages. According to example 2, the macrophages are transduced by a viral vector containing a sequence specifically expressed in activated macrophages, and a leader peptide flanking in 5′ the gene coding the neurotrophic factor such as CNTF.

Some injected macrophages are going through the blood brain barrier reaching suffering motoneurons. They deliver locally the neurotrophic factor allowing motoneurons to survive. The very rapid clinical evolution of the ALS disease is blocked by the treatment which can be renewed.

References

Acsadi G. et al., Nature 1991; 352: 815-818.

Fassati A. et al., Human Gene Therapy 1996; 7(5): 595-602.

Parrish E. P. et al., Gene Therapy 1996; 3: 13-20.

Quantin B. et al., Proc. Natl. Acad. Sci. 1992; 89: 2581-2584.

Ragot T. et al., Nature 1993; 361: 647-650.

Vincent N. e al. Nature genetics 1993; 5: 130-134.

Wolff J. A. e al., Science 1990; 245: 1465-1468.

1. A method for preferentially delivering a therapeutic agent or a diagnostic agent to a central nervous system (CNS) lesion, comprising administering to a patient having or suspected of having a CNS lesion an effective amount of exogenous monocyte derived cells, said monocyte derived cells being loaded with a therapeutic agent with respect to said CNS lesion or a diagnostic marker, and with said monocyte derived cells having the properties of mobilisation towards chemotactic factors released at or adjacent a CNS lesion, thereby to target cells present in the vicinity of said released chemotactic factors.

2. The method according to claim 1, wherein said monocyte derived cells are loaded with a therapeutic agent selected from the group consisting of ciliary neurotrophic factor, brain derived neurotrophic factor, glial cells derived neurotrophic factor, and tyrosine hydroxylase and DOPA carboxylase.

3. The method according to claim 1, wherein the corrective agent is a chemical product.

4. The method according to claim 1, wherein the chemotactic factors are released either by injured or pathological sites spontaneously resulting from said CNS lesion or subsequent to a chemical or physical stimulation of the sites to be treated.

5. The method according to claim 1, wherein the therapeutic agent is selected from the group consisting of ciliary neurotrophic factor, glial cells derived neurotrophic factor, and elements liable to inhibit or to kill abnormally stimulated cells, responsible for or resulting from said CNS lesion.

6. The method of claim 1, wherein said CNS lesion is selected from those causing a disorder selected from the group consisting of adrenoleukodystrophy, spinal muscular atrophy, Gaucher disease, Huntington disease, Alzheimer disease, Parkinson disease, amyotrophic lateral sclerosis, multiple sclerosis, strokes, glioblastoma, cerebral metastasis, infection of the central nervous system, Duchenne disease, Becker disease, muscular dystrophies, neuropathies and muscular necrosis from different origins (including trauma), rheumatoid arthritis, atheromatosis, bone trauma or bone infection or degenerescence, and pulmonary fibrosis.

7. The method of claim 1, wherein said CNS lesion to be treated is selected from those causing a disorder selected from the group consisting of Alzheimer disease, Parkinson disease, amyotrophic lateral sclerosis, multiple sclerosis, and strokes.
